(19) United States
(12) Patent Application Publication
(10) Pub. No.: US 2011/0229540 A1
(43) Pub. Date: Sep. 22, 2011

(54) COSMETIC FORMULATIONS COMPRISING POROUS SILICON

(76) Inventors: Leigh Canham, Worcestershire (GB); Tanya Monga, Worcestershire (GB)

(21) Appl. No.: 13/121,871

(22) PCT Filed: Sep. 30, 2009
(86) PCT No.: PCT/GB2009/051282

§ 371 Date: Jun. 2, 2011

(30) Foreign Application Priority Data
Sep. 30, 2008 (GB) 0817938.4

(51) Int. Cl. A61K 8/25; A61K 8/02; A61K 8/42; A61K 8/67; A61Q 19/00; C01B 33/02; C01B 33/04; C01B 33/113; B32B 3/26

(52) U.S. Cl. 424/401; 514/625; 514/725; 424/600; 423/348; 423/347; 423/325; 428/304.4

(57) ABSTRACT

A cosmetic formulation comprising porous silicon is described.

### FIELD OF THE INVENTION

This invention relates to the use of porous silicon in cosmetic formulations, methods for the production of said formulations and uses of the formulations.

### BACKGROUND OF THE INVENTION

Cosmetic formulations generally refer to substances or preparations intended for placement in contact with an external part of the human body with a view to providing one or more of the following functions: changing its appearance, altering the odour, cleansing, maintaining/improving the condition, perfuming and protecting.

More specific functions provided by cosmetic formulations relate to the following aspects: anti-ageing/anti-wrinkle, anti-acne/pimples/blackheads, cellulite reduction, oedema reduction, moisturising/lubricating, sebum removal, anti-clogging of pores, exfoliation/peeling, colouring/tanning, maintenance via nutrition.

In the cosmetics industry, numerous methods are used to stabilise various ingredients in cosmetic formulations and to control the timing and release of said ingredients. Such methods enable the protection of various ingredients and may facilitate the masking or preservation of aromas. Suitable methods of protection also increase the stability of vitamin or mineral supplements which are normally sensitive to light, UV radiation, metals, humidity, temperature and oxygen.

A formulation which is of use in connection with a particular area of the body may not necessarily be suitable for use on other areas of the body. There are particular challenges in developing and tailoring cosmetic formulations which are suitable for use in some areas of the body, such as the face and neck. There is also the additional challenge that cosmetic compositions for use on the face may be required to impart a significant visual change when compared to cosmetics for use on other parts of the human body As such, there is a continued need for alternative and preferably improved cosmetic formulations for use in connection with the human face which are capable of providing a number of functions.

The present invention is based partly on the surprising finding that porous silicon may be used in cosmetic compositions suitable for use on the human face and, optionally, for the effective and controlled delivery of active ingredients.

### SUMMARY OF THE INVENTION

According to a first aspect of the present invention, there is provided a cosmetic composition for use on the human face comprising porous silicon.

According to a second aspect of the present invention, there is provided a production process for said cosmetic composition according to the first aspect of the present invention, comprising blending said porous silicon and other components of the cosmetic composition.

According to a third aspect of the present invention, the use of porous silicon for delivering an active ingredient to the human face is provided.

According to a further aspect of the present invention, a method is provided of treating and/or cleaning the human face comprising applying a composition according to the first aspect of the present invention to the human face. Methods of treating and/or preventing any one of acne, oily skin, wrinkles, psoriasis on the face are provided. The method may be a non-medical (i.e. “cosmetic”) or medical treatment method. The present invention extends to compositions for use in the prevention and/or treatment of one or more of acne, oily skin, wrinkles, psoriasis, skin blemishes such as birthmarks, scars, moles, blackheads, freckles, pimples, bags or dark circles under the eyes, rosacea, sebhorrhoeic dermatitis, enlarged pores, pitting, enlarged blood vessels, senile freckles on the face.

The porous silicon may comprise at least one ingredient for delivery to the face. Suitable ingredients include one or more of: antioxidants, anti-ageing actives, nutrients, skin lightening agents, moisturisers, antimicrobials, fragrances, oils, vitamins, structural agents, natural actives. The porous silicon may be loaded with the ingredient which may be entrapped in the silicon pores.

The use of porous silicon containing cosmetic formulations according to the present invention seeks to provide one or more of the following: targeted delivery of ingredients; extended release of ingredients including burst fragrance release, for example, during washing; improved bioavailability of actives, including hydrophobic actives; skin exfoliation; sebum absorption/removal; beneficial degradation products such as orthosilicic acid; retention of significant levels of active ingredients on the face over extended periods of time, excellent skin feel and visual appearance.

### DETAILED DESCRIPTION OF THE INVENTION

Porous Silicon

As used herein, and unless otherwise stated, the term “silicon” refers to solid elemental silicon. For the avoidance of doubt, and unless otherwise stated, it does not include silicon-containing chemical compounds such as silica, silicates or silicones, although it may be used in combination with these materials.

The physical forms of porous silicon which are suitable for use in the present invention may be chosen from or comprise amorphous silicon, single crystal silicon and polycrystalline silicon (including nanocrystalline silicon, the grain size of which is typically taken to be 1 to 100 nm) and including combinations thereof. The silicon may be surface porosified, for example, using a stain etch method or more substantially porosified, for example, using an anodisation technique. Following porosification some non-porosified silicon, such as bulk silicon, may be present with the porous silicon. The porous silicon is advantageously selected from microporous and/or mesoporous silicon. Mesoporous silicon contains pores having a diameter in the range of 2 to 50 nm. Microporous silicon contains pores possessing a diameter less than 2 nm.

The average pore diameter is measured using a known technique. Mesopore diameters are measured by very high resolution electron microscopy. This technique and other suitable techniques which include gas-adsorption-desorption analysis, small angle x-ray scattering, NMR spectroscopy or thermoporometry, are described by R. Herino in “Properties of Porous Silicon”, chapter 2.2, 1997. Micropore diameters are measured by xenon porosimetry, where the Xe¹²⁹ nmr signal depends on pore diameter in the sub 2 nm range.

The porous silicon may have a BET surface area of 50 m²/g to 800 m²/g, for example, 100 m²/g to 500 m²/g. The BET surface area is determined by a BET nitrogen adsorption method as described in Brunauer et al., J. Am. Chem. Soc., 60, p309, 1938. The BET measurement is performed using an Accelerated Surface Area and Porosimetry Analyser (ASAP 2400) available from Micromeritics Instrument Corporation, Norcross, Ga. 30093. The sample is outgassed under vacuum at 350° C. for a minimum of 2 hours before measurement.

The purity of the porous silicon may be about 95 to 99.99999% pure, for example about 95 to 99.99% pure. So-called metallurgical silicon which may also be used in the cosmetic compositions has a purity of about 98 to 99.5%. The metallurgical silicon grade silicon preferably has a very low content of all metals (e.g. nickel) known to cause problems in connection with skin hypersensitivity.

Methods for making various forms of silicon which are suitable for use in the present invention are described below. The methods described are well known in the art.

In PCT/GB96/01863, the contents of which are incorporated herein by reference in their entirety, it is described how bulk crystalline silicon can be rendered porous by partial electrochemical dissolution in hydrofluoric acid based solutions. This etching process generates a silicon structure that retains the crystallinity and the crystallographic orientation of the original bulk material. Hence, the porous silicon formed is a form of crystalline silicon. Broadly, the method involves anodising, for example, a heavily boron doped CZ silicon wafer in an electrochemical cell which contains an electrolyte comprising a 20% solution of hydrofluoric acid in an alcohol such as ethanol, methanol or isopropylalcohol (IPA). Following the passing of an anodisation current with a density of about 50 mAcm⁻², a porous silicon layer is produced which may be separated from the wafer by increasing the current density for a short period of time. The effect of this is to dissolve the silicon at the interface between the porous and bulk crystalline regions. Porous silicon may also be made using the so-called stain-etching technique which is another conventional method for making porous silicon. This method involves the immersion of a silicon sample in a hydrofluoric acid solution containing a strong oxidising agent. No electrical contact is made with the silicon, and no potential is applied. The hydrofluoric acid etches the surface of the silicon to create pores.

Mesoporous silicon may be generated from a variety of non-porous silicon powders by so-called “electroless electrochemical etching techniques”, as reviewed by K. Kolasinski in Current Opinions in Solid State & Materials Science 9, 73 (2005). These techniques include “stain-etching”, “galvanic etching”, “hydrothermal etching” and “chemical vapour etching” techniques. Stain etching results from a solution containing fluoride and an oxidant. In galvanic or metal-assisted etching, metal particles such as platinum are also involved. In hydrothermal etching, the temperature and pressure of the etching solution are raised in closed vessels. In chemical vapour etching, the vapour of such solutions, rather than the solution itself is in contact with the silicon. Mesoporous silicon can be made by techniques that do not involve etching with hydrofluoric acid. An example of such a technique is chemical reduction of various forms of porous silica as described by Z. Bao et al in Nature vol. 446 8th March 2007 p172-175 and by E. Richman et al. in Nano Letters vol. 8(9) p3075-3079 (2008). If this reduction process does not proceed to completion then the mesoporous silicon contains varying residual amounts of silica.

Following its formation, the porous silicon may be dried. For example, it may be supercritically dried as described by Canham in Nature, vol. 368, (1994), pp133-135. Alternatively, the porous silicon may be freeze dried or air dried using liquids of lower surface tension than water, such as ethanol or pentane, as described by Bellet and Canham in Adv. Mater, 10, pp487-490, 1998.

Silicon hydride surfaces may, for example, be generated by stain etch or anodisation methods using hydrofluoric acid based solutions. When the silicon, prepared, for example, by electrochemical etching in HF based solutions, comprises porous silicon, the surface of the porous silicon may or may not be suitably modified in order, for example, to improve the stability of the porous silicon in the hair care composition. In particular, the surface of the porous silicon may be modified to render the silicon more stable in alkaline conditions. The surface of the porous silicon may include the external and/or internal surfaces formed by the pores of the porous silicon.

In certain circumstances, the stain etching technique may result in partial oxidation of the porous silicon surface. The surfaces of the porous silicon may therefore be modified to provide: silicon hydride surfaces; silicon oxide surfaces wherein the porous silicon may typically be described as being partially oxidised; or derivatised surfaces which may possess Si—O—C bonds and/or Si—C bonds. Silicon hydride surfaces may be produced by exposing the porous silicon to HF.

Silicon oxide surfaces may be produced by subjecting the silicon to chemical oxidation, photochemical oxidation or thermal oxidation, as described for example in Chapter 5.3 of Properties of Porous Silicon (edited by L. T. Canham, IEE 1997). PCT/GB02/03731, the entire contents of which are incorporated herein by reference, describes how porous silicon may be partially oxidised in such a manner that the sample of porous silicon retains some porous silicon in an unoxidised state. For example, PCT/GB02/03731 describes how, following anodisation in 20% ethanoic HF, the anodised sample was partially oxidised by thermal treatment in air at 500° C. to yield a partially oxidised porous silicon sample.

Following partial oxidation, an amount of elemental silicon will remain. The silicon particles may possess an oxide content corresponding to between about one monolayer of oxygen and a total oxide thickness of less than or equal to about 4.5 nm covering the entire silicon skeleton. The porous silicon may have an oxygen to silicon atomic ratio between about 0.04 and 2.0, and preferably between 0.60 and 1.5. Oxidation may occur in the pores and/or on the external surface of the silicon.

Derivatised porous silicon is porous silicon possessing a covalently bound monolayer on at least part of its surface. The monolayer typically comprises one or more organic groups that are bonded by hydrosilylation to at least part of the surface of the porous silicon. Derivatised porous silicon is described in PCT/GB00/01450, the contents of which are incorporated herein by reference in their entirety. PCT/GB00/01450 describes derivatisation of the surface of silicon using methods such as hydrosilyation in the presence of a Lewis acid. In that case, the derivatisation is effected in order to block oxidation of the silicon atoms at the surface and so stabilise the silicon. Methods of preparing derivatised porous silicon are known to the skilled person and are described, for example, by J. H. Song and M. J. Sailor in Inorg. Chem. 1999, vol 21, No.

1-3, pp 69-84 (Chemical Modification of Crystalline Porous Silicon Surfaces). Derivitisation of the silicon may be desirable when it is required to increase the hydrophobicity of the silicon, thereby decreasing its wettability. Preferred derivatised surfaces are modified with one or more alkyne groups. Alkyne derivatised silicon may be derived from treatment with acetylene gas, for example, as described in “Studies of thermally carbonized porous silicon surfaces” by J. Salonen et at in Phys Stat. Solidi (a), 182, pp123-126, (2000) and “Stabilisation of porous silicon surface by low temperature photoassisted reaction with acetylene”, by S. T. Lakshmikumar et al in Curr. Appl. Phys. 3, pp185-189 (2003). Mesoporous silicon may be derivatised during its formation in I-IF-based electrolytes, using the techniques described by G. Mattel and V. Valentini in Journal American Chemical Society vol. 125, p9608 (2003) and Valentini et al. Physica Status Solidi (c) 4 (6) p2044-2048 (2007).

The surface chemistry of the porous silicon may be adapted depending on the particular application.

The porous silicon may also comprise a capping layer in order to prevent release of the loaded ingredient prior to application to the human face too soon following application. In particular, the porous silicon may be capped using ultrathin capping layers or beads around the loaded porous silicon. The capping layers may provide retention of the loaded ingredient over a number of months of storage in liquid media, for example from about 1 year up to about 5 years. After the container has been opened, retention may be for a shorter period but may still be up to about 1 year after opening. The capping layer may also be designed to trigger active release of the loaded ingredient through site-specific degradation when in contact with the human face. Suitable capping materials include one or more of carbohydrates, gums, lipids, proteins, celluloses, synthetic polymers, synthetic elastomers, inorganic materials. The capping layer may also serve to improve dispersion in the cosmetic compositions and the present invention extends to a method for dispersing capped porous silicon in the compositions described herein. The thickness of the capping layer may be about 0.1 to 50 μm in thickness, for example about 1 to 10 μm, for example about 1 to 5 μm.

The thickness of the capping layer is measured by mechanically fracturing a number of the capped particles and examining their cross-sectional images in a high resolution scanning electron microscope, equipped with energy dispersive x-ray analysis (EDX analysis) of chemical composition. Alternatively, if the particle size distributions are measured accurately, before and after capping, then the average thickness of micron thick layer caps can be estimated. For relatively narrow particle size distributions and uniform coatings, if the density of the capping layer is known accurately, then accurate gravimetric measurements of weight increase that accompanies capping can also yield an average cap thickness.

Suitable examples of carbohydrates include starch, dextran, sucrose, corn, syrup. Suitable examples of gums include carrageenan, sodium alginate, gum Arabic, agar. Suitable examples of lipids include fats, hardened oils, paraffin, stearic acid, wax, diglycerides, monoglycerides. Suitable examples of proteins include albumin, casein, gluten, gelatine. Suitable examples of celluloses include carbomethylcellulose, acetylcellulose, methylcellulose. Suitable examples of polymers include synthetic polymers such as polyacrylate, polyethylene, polystyrene, polyvinyl alcohol, polyurea. Suitable examples of elastomers include acrylonitrile, polybutadience. Suitable examples of inorganic materials include calcium sulphate, silicates, clays, silicon, silicon dioxide, calcium phosphate. The capping layer may comprise, consist of, or consist essentially of elemental silicon, for example, in the form of an amorphous silicon coating or a discontinuous layer of silicon nanoparticles. The loaded ingredient and the capping layer may be the same.

Suitable methods for capping the porous silicon include spray drying, fluidized bed coating, pan coating, modified microemulsion techniques, melt extrusion, spray chilling, complex coacervation, vapour deposition, solution precipitation, emulsification, supercritical fluid techniques, physical sputtering, laser ablation, and thermal evaporation. The capping layer, may for example be degraded by a sudden increase in temperature, such as that provided by warm water. The capping layer may comprise two overlying distinct capping layers, with each layer possessing different properties.

Spray drying techniques are usually carried out from aqueous feed formulations, in which case the capping layer should be soluble in water at an acceptable level. Typical materials include gum acacia, maltodextrins, hydrophobically modified starch and mixtures thereof. Other polysaccharides such as alginate, carboxymethylcellulose, guar gum and proteins such as whey proteins, soy proteins, sodium caseinate are also suitable. Aqueous two phase systems (ATPs) which may result from the phase separation of a mixture of soluble polymers in a common solvent due to the low entropy of mixing of polymer mixtures can be used to design double encapsulated ingredients in a single spray drying step.

Spray chilling or cooling is generally considered one of the least expensive encapsulation technologies. This technique may also be referred to as matrix encapsulation. It is particularly suitable for encapsulating organic and inorganic materials as well as textural ingredients, enzymes, flavours and other ingredients to improve heat stability. Matrix encapsulation may lead to some of the loaded ingredient being incorporated in the capping layer.

Extrusion is suitable for the encapsulation of volatile and unstable flavours. This process is suitable for imparting long shelf life to normally oxidation prone compounds.

Coacervation is particularly useful in connection with the use of high levels of loaded ingredient and is typically used for encapsulating nutrients, vitamins, preservatives, enzymes. Coacervation requires the phase separation of one or many hydrocolloids from solution and the subsequent deposition of the newly formed coacervate phase around the porous material which is suspended or emulsified in the same reaction media. The hydrocolloid shell may then be crosslinked using an appropriate chemical or enzymatic crosslinker if required.

When the capping layer includes elemental silicon, the amorphous silicon coating may be deposited by physical sputtering and may have a thickness of 500 nm to 5 μm. The silicon nanoparticles are preferably bound to the porous silicon by solution based techniques.

There are various mechanisms by which the release of the loaded ingredient may be triggered. These are:

(a) Biodegradation

The capping layer may be degraded by enzymes or bacteria present at the intended site of use (active release).

(b) Mechanical

The capping layer may be degraded by mechanical forces at the intended site of use, such as frictional forces upon application of the cosmetic formulation.

(c) Thermal

The capping layer may be degraded by a sudden increase of temperature such as exposure to body temperature (37° C.) or warm water (25 to 55° C.).

(d) Optical Irradiation

The capping layer may be degraded by exposure to sunlight or UV from commercial tanning equipment.

(e) Chemical Environment

The capping layer may be degraded by a change in the chemical environment, such as a pH change from acidic to alkali or vice versa.

Particulate Silicon

The silicon is typically present in particulate form. Methods for making silicon powders such as silicon microparticles and silicon nanoparticles are well known in the art. Silicon microparticles are generally taken to mean particles of about 1 to 1000 μm in diameter and silicon nanoparticles are generally taken to mean particles possessing a diameter of about 100 nm and less. Silicon nanoparticles therefore typically possess a diameter in the range of about 1 nm to about 100 nm, for example about 5 nm to about 100 nm. Fully biodegradable mesoporous silicon typically has an interconnected silicon skeleton with widths in the 2-5 nm range. In connection with the present invention, mesoporous silicon particles possessing a diameter of 50 nm-1000 nm, for example 100-500 nm may be employed. However, advantageously, the porous silicon particles have a diameter of 5 μm to 250 μm, more particularly 10 μm to 150 μm for example 20 μm to 60 μm. Methods for making silicon powders are often referred to as “bottom-up” methods, which include, for example, chemical synthesis or gas phase synthesis. Alternatively, so-called “top-down” methods refer to such known methods as electrochemical etching or comminution (e.g. milling as described in Kerkar et al. J. Am. Ceram. Soc., vol. 73, pages 2879-2885, 1990.). PCT/GB02/03493 and PCT/GB01/03633, the contents of which are incorporated herein by reference in their entirety, describe methods for making particles of silicon, said methods being suitable for making silicon for use in the present invention. Such methods include subjecting silicon to centrifuge methods, or grinding methods. Porous silicon powders may be ground between wafers or blocks of crystalline silicon. Since porous silicon has lower hardness than bulk crystalline silicon, and crystalline silicon wafers have ultrapure, ultrasmooth surfaces, a silicon wafer/porous silicon powder/silicon wafer sandwich is a convenient means of achieving for instance, a 1-10 μm particle size from much larger porous silicon particles derived, for example, via anodisation.

The shape of the porous silicon particles may also be tailored for specific applications. For example, the silicon particles may be spheroidised in order to provide a so-called silky feel. Spheroidisation may be achieved by using a plasma process followed by stain-etching. A suitable system comprises a plasma torch mounted on a reactor vessel. The silicon powder feed is passed into the plasma to vaporise the powder; the equivalent temperature (about 10,000 K) is dependant on feed stock size, flow rate and material properties. The hot silicon vapour is cooled rapidly in a gas quenching region of the reactor, before passing into a cyclone for coarse powder separation. The remaining solidified powder passes into a collection filter for recovery as product. Material may be recovered from either the filter or cyclone depending on the requirement, but typically, cyclone material tends to be spherodised micron sized particles and the filter material, fine nanomaterial (5-100 nm nominal particle size). The spherodized microparticles may be created from molten droplets solidifying in the reactor and centrifuging out in the cyclone. A suitable feed rate is typically approximately 200 g/hr, 22 to 30 kW using a non transferred plasma source utilising argon primary gas and without secondary gas. The system is typically fully inerted and run at positive pressure to minimise oxygen ingression. Argon may be used as a quench gas at, for example, 800 Slpm (Standard litres per minutes).

The surface of silicon particles prepared by “top down” or “bottom up” methods may also be a hydride surface, a partially oxidised surface, a fully oxidised surface or a derivatised surface. Milling in an oxidising medium such as water or air will result in silicon oxide surfaces. Milling in an organic medium may result in, at least partial derivatisation of the surface. Gas phase synthesis, such as from the decomposition of silane, will result in hydride surfaces. The surface may or may not be suitably modified in order, for example, to improve the stability of the particulate silicon in the cosmetic composition.

Other examples of methods suitable for making silicon nanoparticles include evaporation and condensation in a subatmospheric inert-gas environment. Various aerosol processing techniques have been reported to improve the production yield of nanoparticles. These include synthesis by the following techniques: combustion flame; plasma; laser ablation; chemical vapour condensation; spray pyrolysis; electrospray and plasma spray. Because the throughput for these techniques currently tends to be low, preferred nanoparticle synthesis techniques include: high energy ball milling; gas phase synthesis; plasma synthesis; chemical synthesis; sonochemical synthesis.

Some methods of producing silicon nanoparticles are described in more detail below.

High-Energy Ball Milling

High energy ball milling, which is a common top-down approach for nanoparticle synthesis, has been used for the generation of magnetic, catalytic, and structural nanoparticles, see Huang, “Deformation-induced amorphization in ball-milled silicon”, Phil. Mag. Lett., 1999, 79, pp305-314. The technique, which is a commercial technology, has traditionally been considered problematic because of contamination problems from ball-milling processes. However, the availability of tungsten carbide components and the use of inert atmosphere and/or high vacuum processes has reduced impurities to acceptable levels. Particle sizes in the range of about 0.1 to 1 μm are most commonly produced by ball-milling techniques, though it is known to produce particle sizes of about 0.01 μm.

Ball milling can be carried out in either “dry” conditions or in the presence of a liquid, i.e. “wet” conditions. For wet conditions, typical solvents include water or alcohol based solvents.

Gas Phase Synthesis

Silane decomposition provides a very high throughput commercial process for producing polycrystalline silicon granules. Although the electronic grade feedstock (currently about $30/kg) is expensive, so called “fines” (microparticles and nanoparticles) are a suitable waste product for use in the present invention. Fine silicon powders are commercially available. For example, NanoSi™ Polysilicon is commercially available from Advanced Silicon Materials LLC and is a fine silicon powder prepared by decomposition of silane in a hydrogen atmosphere. The particle size is 5 to 500 nm and the BET surface area is about 25 m²/g. This type of silicon has a tendency to agglomerate, reportedly due to hydrogen bonding and Van der Waals forces.

Plasma Synthesis

Plasma synthesis is described by Tanaka in “Production of ultrafine silicon powder by the arc plasma method”, J. Mat. Sci., 1987, 22, pp2192-2198. High temperature synthesis of a range of metal nanoparticles with good throughput may be achieved using this method. Silicon nanoparticles (typically 10-100 nm diameter) have been generated in argon-hydrogen or argon-nitrogen gaseous environments using this method.

Chemical Synthesis

Solution growth of ultra-small (<10 nm) silicon nanoparticles is described in US 20050000409, the contents of which are incorporated herein in their entirety. This technique involves the reduction of silicon tetrahalides such as silicon tetrachloride by reducing agents such as sodium napthalenide in an organic solvent. The reactions lead to a high yield at room temperature.

Sonochemical Synthesis

In sonochemistry, an acoustic cavitation process can generate a transient localized hot zone with extremely high temperature gradient and pressure. Such sudden changes in temperature and pressure assist the destruction of the sonochemical precursor (e.g., organometallic solution) and the formation of nanoparticles. The technique is suitable for producing large volumes of material for industrial applications. Sonochemical methods for preparing silicon nanoparticles are described by Dhas in “Preparation of luminescent silicon nanoparticles: a novel sonochemical approach”, Chem. Mater., 10, 1998, pp 3278-3281.

Mechanical Synthesis

Lam et al have fabricated silicon nanoparticles by ball milling graphite powder and silica powder, this process being described in J. Crystal Growth 220(4), p466-470 (2000), which is herein incorporated by reference in its entirety. Arujo-Andrade et al have fabricated silicon nanoparticles by mechanical milling of silica powder and aluminum powder, this process being described in Scripta Materialia 49(8), p773-778 (2003).

An alternative method for making porous silicon from nanoparticles includes exposing nanoparticulate elemental silicon to a pulsed high energy beam. The high energy beam may be a laser beam or an electron beam or an ion beam. Preferably, the high energy beam creates a condition wherein the elemental silicon is rapidly melted, foamed and condensed. Preferably, the high energy beam is a pulsed laser beam.

In the present invention, particle size distribution measurements, including the mean particle size (d₅₀/μm) of the porous silicon particles are measured using a Malvern Particle Size Analyzer, Model Mastersizer, from Malvern Instruments. A helium-neon gas laser beam is projected through a transparent cell which contains the silicon particles suspended in an aqueous solution. Light rays which strike the particles are scattered through angles which are inversely proportional to the particle size. The photodetector array measures the quantity of light at several predetermined angles. Electrical signals proportional to the measured light flux values are then processed by a microcomputer system, against a scatter pattern predicted from theoretical particles as defined by the refractive indices of the sample and aqueous dispersant to determine the particle size distribution of the silicon.

Ingredients

The porous silicon may be loaded with one or more active ingredients. These ingredients include one or more of the following: antioxidants, anti-ageing actives, skin lightening agents, nutrients, moisturisers, antimicrobials, sunscreens, fragrances, oils, vitamins, structural agents, natural actives.

Suitable antioxidant agents include pycnogenol, plant and fruit extracts, marine extracts, ascorbic acid, glucosides, vitamin E, herbals extracts and synergistic combinations thereof. Suitable anti ageing actives include ceramide, peptides, plant extracts, marine extracts, collagen, calcium amino acids vitamin A, vitamin C and CoQ10. Suitable skin lightening agents include liquorice, arbutin, vitamin C, kojic acid.

Suitable moisturisers include panthenol, amino acids, hyaluronic acids, ceramides, sodium PCS, glycerols and plant extracts. Vitamin A may be present in one or more of its various forms. For example vitamin A may be present as retinol.

The ingredient to be loaded with the porous silicon may be dissolved or suspended in a suitable solvent, and porous silicon particles may be incubated in the resulting solution for a suitable period of time. Both aqueous and non-aqueous slips have been produced from ground silicon powder and the processing and properties of silicon suspensions have been studied and reported by Sacks in Ceram. Eng. Sci. Proc., 6, 1985, pp1109-1123 and Kerkar in J. Am. Chem. Soc. 73, 1990, pp2879-85. The wetting of solvent will result in the ingredient penetrating into the pores of the silicon by capillary action, and, following solvent removal, the ingredient will be present in the pores. Preferred solvents are water, ethanol, and isopropyl alcohol, GRAS solvents and volatile liquids amenable to freeze drying.

In general, if the ingredient to be loaded has a low melting point and a decomposition temperature significantly higher than that melting point, then an efficient way of loading the ingredient is to melt the ingredient.

Higher levels of loading, for example, at least about 15 wt % of the loaded ingredient based on the loaded weight of the silicon may be achieved by performing the impregnation at an elevated temperature. For example, loading may be carried out at a temperature which is at or above the melting point of the ingredient to be loaded. Quantification of gross loading may conveniently be achieved by a number of known analytical methods, including gravimetric, EDX (energy-dispersive analysis by x-rays), Fourier transform infra-red (FTIR), Raman spectroscopy, UV spectrophotometry, titrimetric analysis, HPLC or mass spectrometry. If required, quantification of the uniformity of loading may be achieved by techniques that are capable of spatial resolution such as cross-sectional EDX, Auger depth profiling, micro-Raman and micro-FTIR.

The loading levels can be determined by dividing the volume of the ingredient taken up during loading (equivalent to the mass of the ingredient taken up divided by its density) by the void volume of the porous silicon prior to loading multiplied by one hundred.

Cosmetic Compositions

Cosmetic compositions suitable for use on the face in accordance with the present invention may be in the form of creams, pastes, serums, gels, lotions, oils, milks, stick, ointments, powder (including dry powder), solutions, suspensions, dispersions and emulsions.

The porous silicon may be present in an amount of from 0.01 wt % to 40 wt % based on the total weight of the cosmetic composition for example 0.1 to 10 wt %.

Suitable cosmetic compositions include: foundation, mascara, lipstick, lip balm, lip gloss, colour cosmetics, face cream, eye cream, after-shave, toner, cleanser, aftersun, moisturiser, face masks, lip and eye liners, shaving cream, face powder (loose and pressed), eye shadow, bronzer, blush, concealers, face scrub and make up removers. The components comprised in these compositions are well known to the skilled person and these components are suitable for use in the present invention. These components may include a vehicle to act as a carrier or dispersant, emollients, thickeners, opacifiers, perfumes, colour pigments, skin feel components, other sebum absorbing materials, preservatives, mineral fillers and extenders, colour pigments.

In general, the cosmetic compositions may contain a vehicle to act as a carrier or dispersant for the porous silicon so as to facilitate the distribution of the porous silicon when the composition is applied to the skin. Vehicles other than, or in addition to water can include cosmetic astringents, liquid or solid emollients, emulsifiers, film formers, humectants, skin protectants, solvents, propellants, skin-conditioning agents, solubilising agents, suspending agents, surfactants, ultraviolet light absorbers, waterproofing agents, viscosity increasing agents, waxes, wetting agents. The carrier or dispersant may form about 50 to 90 wt % of the composition. An oil or oily material may be present to provide a water in oil or oil in water emulsion. The compositions may contain at least one active ingredient including skin colourants, drug substances such as anti-inflammatory agents, antiseptics, antifungals, steroids or antibiotics.

Levels of emollients may be 0.5 wt % to 50 wt %, for example 5 to 30 wt %. General classes of emollients include esters, fatty acids, alcohols, polyols, hydrocarbons. Examples of esters include dibutyl adipate, diethyl sebacate, lauryl palmitate. Suitable alcohols and acids include those having from 10 to 20 carbon atoms, for example cetyl, myristyl, palmitic and stearyl alcohols and acids. Examples of polyols include propylene glycol, sorbitol, glycerine. Suitable hydrocarbons include those possessing 12 to 30 carbon atoms, e.g. mineral oil, petroleum jelly, squalene.

A thickener may be present in levels from 0.1 to 20 wt %, for example about 0.5 to 10 wt %. Examples of suitable thickeners include gums e.g. xanthan, carrageenan, gelatin. Alternatively, the thickening function may be provided by any emollient which is present.

Suitable mineral fillers or extenders include chalk, talc, kaolin, mica.

Other minor components may be incorporated into the cosmetic compositions, such as skin feel components. Skin feel components may also include colouring agents, opacifiers and perfumes. These minor components may range from 0.001 wt % to 10 wt %.

Other suitable ingredients may include sebum absorbing materials (other than porous silicon) such as starch, colour pigments, e.g. iron oxides, preservatives such s trisodium EDTA. Other minor components include colouring agents, perfumes, opacifiers which may range from 0.01 to 10 wt %.

Lipstick typically contains pigments, oils, waxes, and emollients and applies colour and texture to the lips. Lip balm is a substance topically applied to the lips of the mouth to relieve chapped or dry lips. Lip gloss is topically applied to the lips of the mouth, but generally has only cosmetic properties. Lip balm may be manufactured from beeswax, petroleum jelly, menthol, camphor, scented oils, and various other ingredients. Other ingredients such as vitamins, alum, salicyclic acid or aspirin may also be present. The primary purpose of lip balm is to provide an occlusive layer on the lip surface to seal moisture in lips and protect them from external exposure. The occlusive materials like waxes and petroleum jelly prevent moisture loss and maintain lip comfort while flavourants, colorants, sunscreens and various medicaments can provide additional, specific benefits. Lip balm usually comes in containers for application with the fingers or in stick form which is applied directly to the lips.

Mascaras can broadly be divided in two groups: water resistant mascaras (often labelled waterproof) and non-water resistant mascaras. Water resistant mascaras have a composition based on a volatile solvent (e.g. isododecane), animal-derived waxes (e.g. beeswax), vegetal based waxes (e.g. carnauba wax, rice bran wax, candelila wax), mineral origin wax (ozokerite, paraffin), pigments (e.g. iron oxide, ultramarine) and film forming polymers. These mascaras do not contain water-sensitive moieties and afford resistance to tears, sweat or rain. Non water-resistant mascaras are based on water, soft surfactants (e.g. triethanolamine stearate), animal-derived waxes (e.g. beeswax), vegetal based waxes (e.g. rice bran wax, candelilla wax), mineral origin waxes (ozokerite, paraffin), pigments (iron oxide, ultramarine), thickening polymers (gum arabic, hydrophobically modified cellulose) and preservatives. These mascaras can run under the effect of tears, but are easily removed with soap and water. Polymers in a water dispersed form (latexes) can bring some level of water resistance to the group of normally non-water resistant mascaras. Waterproof mascaras are similar to oil-based or solvent-based paints. Non water-resistant mascaras behave like water based paints. For intermediate water sensitivity, mascaras contain polymer dispersions.

Face powder is typically applied to the face to set foundation after application. It is absorbent and provides toning to the skin. It can also be reapplied throughout the day to minimize shininess caused by oily skin. There is translucent sheer powder, and there is pigmented powder. Certain types of pigmented facial powders are meant to be worn alone with no base foundation. Powder tones the face and gives an even appearance. Besides toning the face, some SPF based powders can also reduce skin damage from the sun and environmental stress. It comes packaged either as a compact or as loose powder. It can be applied with a sponge, brush, or powder puff. Due to the wide variation among human skin tones, there is a corresponding variety of colours of face powder. There are also several types of powder. A common powder used in beauty products is talc. Some commercially available brands may contain natural mineral ingredients. Such products are promoted as being safe and calming for rosacea, as well as improving wrinkles and skin that has been over exposed to sun and has hyper pigmentation. Powdering is a very popular cosmetic technique and is used by many people.

### EXAMPLES

The invention will now be described by way of example only with reference to the following examples.

### Example 1

Silicon microparticles are spherodised using a high temperature plasma process. The spherodized microparticles are created from molten droplets solidifying in a reactor and centrifuging out in a cyclone. A feed rate of approximately 200 g/hr, 22 to 30 kW using a non transferred plasma source utilising argon primary gas and without secondary gas is used. The system is fully inerted and run at positive pressure to minimise oxygen ingression. Argon is used as a quench gas at 800 Slpm. The particles are then classified to have a d₅₀ of 10 μm and a d₉₀ of 25 μm. The classified particles are then porosified using stain etching. The active, D-panthenol, a common moisturising agent in cosmetic formulations, is loaded by immersing the mesoporous silicon powder in a bath of the active held at a temperature in the range 75-100° C. for up to 1 hour. For 70% porosity particles, loading levels of up to 40 wt % D-Panthenol are achieved with an excess of active present. By adding an excess of mesoporous silicon, surface D-Panthenol is minimized. Partially loaded microparticles are subsequently capped by immersion in a wax melt held just above its melting point, typically in the range 50-70° C. for up to 15 minutes.

### Example 2

This example describes the use of mesoporous silicon for entrapping and protecting retinol from light induced degradation. Retinol was entrapped in (i) an anodised and (ii) partially oxidised (500° C. and 700° C.) porous silicon membranes. The stability of retinol within the porous silicon was evaluated as a function of time in order to determine the suitability of using porous silicon for improving the long term stability of retinol against light induced degradation. More specifically, the materials used were: (i) an anodised mesoporous silicon membrane possessing 62.9 vol % porosity, (ii) an oxidised (500° C.) mesoporous silicon membrane, (iii) an oxidised (700° C.) mesoporous silicon membrane, (iv) for the purposes of comparison, porous silica powder (Syloid 74FP grade, WR Grace Davison GmbH). Retinol was obtained from Fluka. The apparatus used for conducting measurements was a UV-visible Spectrophotometer (Thermo Fisher UV10) and a UV Lamp (Ultraviolet Products Inc. BLAK-RAY B-100A).

A stock solution of retinol (1 mg/ml) was prepared in ethanol under low light conditions and absorption scans were performed on the UV-visible spectrophotometer from 200-500 nm. In addition, various solutions of retinol prepared in ethanol with concentrations ranging from 1 μg/ml to 10 μg/ml were prepared via a serial dilution method. The solutions were freshly prepared and the UV absorbance at 325 nm was recorded using a Thermo Fisher spectrophotometer (UV10). The absorbance values were plotted against concentration and a linear fit was calculated. A plot of absorbance versus concentration resulted in a linear fit with a linear regression (R²) value of 0.99963 and sensitivity of 1 μg/ml.

Loading Retinol into Anodised Porous Silicon Membranes

The anodised porous silicon membrane (62.9 vol % porosity, 159 μm thickness) was prepared using known techniques. An appropriate amount of retinol (equivalent to a theoretical loading of 40 wt %) was dissolved in 0.1 ml ethanol. The retinol solution was added dropwise onto the membrane under low light conditions. The membrane was then allowed to dry, until all the ethanol evaporated leaving just the retinol in the pores. The weight of the membrane was recorded every 15 min. After the ethanol was evaporated, the surface of the membrane was wiped with cotton buds to remove any retinol which may still have been present. The membrane was then placed in 5 ml of ethanol to allow the retinol to leach out from the pores. The sample was analyzed using UV-Vis spectroscopy to quantify the amount of retinol present.

The weight of the retinol loaded porous silicon membrane remained constant after about 45 min and the final weight was recorded (Table 1). The retinol-loaded porous silicon membrane was placed in ethanol and left on a magnetic stirrer for an hour, to help leach out the retinol and the absorbance was measured at 325 nm. This showed the average amount of active retinol present to be 1.8 mg which is equivalent to 12.24% of the amount of retinol originally loaded.

TABLE 1
Summary of retinol loading in anodised pSi under low light conditions.

| | | | | Amount | |
|---|---|---|---|---|---|
| | | | | of | |
| | | Final | | active | |
| | | weight | Weight | retinol | |
| | Initial | of | of | calculated | Active |
| Mem- | weight | retinol | retinol | from UV | retinol |
| brane | of | loaded | added | absorption | fraction |
| sample | pSi (mg) | pSi (mg) | (mg) | data (mg) | % |
| M₁ | 22.9 | 38.7 | 15.1 | 2.07 | 13.69 |
| M₂ | 21.4 | 35.3 | 14.1 | 1.52 | 10.79 |

pSi - Porous silicon

Loading Retinol into Oxidised Porous Silicon Membranes

Porous silicon membranes oxidised at 500° C. and 700° C. were prepared and loaded with 0.1 ml of retinol solution in ethanol (80 mg/ml). The retinol solution was added dropwise to the membranes to allow the retinol solution to seep into the pores. The final weights of the dried retinol-loaded oxidised membranes were recorded. The retinol loaded into the pores of the oxidised porous silicon membranes was leached out, by placing the membranes in 10 ml of ethanol and leaving on a magnetic stirrer for 30 min. The absorbance of the resulting solution was recorded using a UV-Vis spectrophotometer at 325 nm. The weights of the oxidised membranes at 700° C. before and after loading retinol are shown in Table 2. The difference of the initial weight of the porous silicon membrane from the final weight after solvent removal gives the weight of the retinol present in the membrane.

TABLE 2
Retinol loading in pSi membrane oxidised at 700° C.

| | | | | Amount | |
|---|---|---|---|---|---|
| | | | | of active | |
| | | Final | Weight | retinol | |
| | Initial | weight | of | calculated | Active |
| Mem- | weight | of retinol | retinol | from UV | retinol |
| brane | of pSi | loaded pSi | loaded | absorption | fraction |
| sample | (W₁ mg) | (W₂ mg) | (W₂-W₁ mg) | data (mg) | % |
| M₁ | 17.6 | 24.7 | 7.1 | 1.22 | 17.14 |
| 700° C. | | | | | |
| M₂ | 19.6 | 24.6 | 5.0 | 0.77 | 15.33 |
| 700° C. | | | | | |

pSi - Porous silicon

Loading and Release of Retinol from Porous Silicon Membranes Oxidised at 700° C.

The above loading experiments were repeated with porous silicon membranes partially oxidised at 700° C. The dried retinol-loaded membranes were immersed in 10 ml ethanol and placed on a magnetic stirrer. Aliquots of sample were removed at various times and the absorbance was measured at 325 nm using a UV-Vis spectrophotometer. A retinol solution of known concentration was prepared as a control and kept under the same light conditions (daylight) as the membranes and the UV absorbance was noted at the same time points as the membranes. The weights of the membranes were recorded before and after loading the retinol (Table 3). The loaded membranes were immersed into 20 ml of ethanol and placed on a magnetic stirrer. The retinol was allowed to leach out from the pores into the ethanol. Aliquots of the ethanol were taken and the absorbance was read at 325 nm. The loading level was similar in both membranes.

TABLE 3
Summary of retinol loading in pSi membrane oxidised at 700° C.

| | | | | Amount of active | |
|---|---|---|---|---|---|
| Oxidised | | Final weight | | retinol calculated | Active |
| pSi | | of retinol | Weight of | from UV | retinol |
| Membrane | Initial weight | loaded pSi | retinol loaded | absorption data | fraction |
| 700° C. | of pSi (W₁ mg) | (W₂ mg) | (W₂-W₁ mg) | (mg) | % |
| M₁ | 21.0 | 29.1 | 8.1 | 3.46 | 42.74 |
| M₂ | 17.5 | 25.6 | 8.1 | 3.38 | 41.72 |

pSi - Porous silicon

The membranes were kept in ethanol and the absorbance was read at 1, 2, 3, 4, 5, 21, 27 and 48 h (Table 4). At 30 min, the amount of retinol was marginally greater in the first membrane and this could be attributed to the larger amount of porous silicon in M₁, hence the higher stability of retinol in M₁.

TABLE 4
Amount of retinol recovered as a function of time.

| | Amount of active | Amount of active | Amount of retinol |
|---|---|---|---|
| | retinol calculated | retinol calculated | calculated from UV |
| Time | from UV absorption | from UV absorption | absorption data (mg) |
| (h) | data (mg) M₁ | data (mg) M₂ | Standard solution |
| 0.5 | 3.46 | 3.38 | 3.22 |
| 1 | 3.42 | 3.29 | 3.11 |
| 2 | 3.13 | 3.21 | 2.78 |
| 3 | 2.86 | 3.06 | 2.57 |
| 4 | 2.55 | 3.24 | 2.35 |
| 5 | 3.00 | 2.87 | 2.18 |
| 21 | 2.74 | 2.68 | 1.80 |
| 27 | 2.59 | 2.54 | 1.68 |
| 48 | 2.07 | 2.20 | 1.35 |

From the retinol stock solution used to load the membranes, a solution of known concentration (which was the same as that present in the porous silicon membrane) was made and left under similar conditions (daylight in ambient air at 20° C. ±5° C.) as the membranes. The absorbance was read at 325 nm at the same time points (Table 4). A higher amount of undegraded retinol was recovered from the membrane samples compared to the standard retinol solution for each time point. The porous silicon offers better protection for retinol from light induced degradation when present within the pores of a porous silicon membrane compared to when the retinol is present in ethanol.

From the above experiments (Tables 1 and 2), the average active retinol fraction was 12.2% for the anodised porous silicon membrane (loaded under low light conditions) and increased to an average value of 42.2% for porous silicon membranes oxidised at 700° C. (retinol loading carried out under daylight conditions). Retinol-loaded oxidised porous silicon samples analysed over time showed the presence of retinol after 48 h. The oxidised membrane offered better protection to retinol than the anodised porous silicon membranes. Repeats of the retinol loading in oxidised porous silicon membranes (700° C.) showed a significant increase. This increase may be attributed to the fact that in repeat experiments only a single porous silicon membrane was used while loading in the earlier experiments were done with more samples of membrane allowing for loss of retinol during the extended time taken for loading.

Loading Retinol into Porous Silica

For the purposes of comparison, retinol was loaded into porous silica by adding 0.1 ml of retinol in ethanol solution (80 mg/ml) dropwise. The mixture was allowed to dry and the final weight was recorded. The free flowing powder was kept on the bench top and exposed to daylight. Similarly, porous silicon membrane oxidized at 700° C. was loaded with retinol (0.1 ml of 80 mg/ml stock) and left to dry. After the ethanol had evaporated, the final weight was recorded and the membrane was kept on the bench top and exposed to daylight. Both the retinol loaded silica powder and porous silicon membranes were left for 24 h and then analyzed for active retinol content.

The retinol loaded silica powder sample took longer to dry and changed colour from a dark yellow (immediately after adding retinol solution) to a pale yellow colour (24 h). The retinol was leached out from both samples by placing in a known volume of ethanol and was placed on a magnetic stirrer for 30 min. Aliquots of ethanol were removed and their absorbance was read at 325 nm (Table 5).

TABLE 5
Summary of retinol loading into silica powder and pSi
membrane oxidised at 700° C. and analysed after 24 h.

| | | | | Amount | |
|---|---|---|---|---|---|
| | | | | of active | |
| | | | | retinol | |
| | | Final | | after 24 h | |
| | Initial | weight | Weight of | calculated | Active |
| | weight of | of retinol | retinol | from UV | retinol |
| | pSi (W₁ | loaded pSi | loaded | absorption | fraction |
| Sample | mg) | (W₂ mg) | (W₂-W₁ mg) | data (mg) | (24 h) |
| pSi 700° C. | 14.5 | 22.6 | 8.1 | 2.84 | 32.37 |
| Silica | 15.7 | 24.8 | 9.1 | 0.38 | 4.39 |

pSi - Porous silicon

Although both samples were subjected to similar loading and bench top storage conditions, the retinol was better protected when present in the porous silicon membrane than the silica powder. This is evidenced by the far greater active retinol fraction of 32.37% in the porous silicon membrane compared to 4.39% for the silica powder. The value for the porous silicon membrane compares well with those results presented in Table 4.

Stability of Retinol Entrapped in Porous Silicon, Exposed to Longwave UV Light

Retinol was loaded into an oxidised (700° C.) porous silicon membrane as described earlier in this example. The loaded membranes were allowed to dry and the final weights were recorded. The retinol loaded silicon membrane was placed under a longwave UV lamp (7 μW cm⁻², 365 nm light at 15 cm in air at 40 ±5° C.) for an hour. Retinol loaded silicon membrane covered with aluminum foil was used as a control and placed under the UV lamp. The silicon membranes were then immersed in 10 ml ethanol and placed on a magnetic stirrer. Aliquots of sample were removed and the absorbance was measured at 325 nm. The retinol loaded silicon membranes were exposed to longwave UV light in ambient air for an hour. The temperature of the samples was approximately 40° C. The control sample (covered with aluminum foil) served to protect the membrane from UV light and to a limited extent reflected the heat away. The absorbance scans indicated that in spite of exposure to UV light there was no drastic degradation of retinol. A retinol solution exposed to UV light for an hour showed significant degradation and reduction of peak height in the UV-visible spectrum. However, when retinol loaded porous silicon membrane was exposed to UV light there was no drastic change in the shape of the absorbance curve and the amount of active retinol calculated was considerably higher when compared to the retinol solution.

In summary, it is evident that: loading of retinol into porous silicon was greater in the oxidised porous silicon membrane compared to the anodised porous silicon membrane; mesoporous silicon offers significantly greater UV protection to loaded retinol than when loaded into mesoporous silica powder and a separate retinal solution.

### Example 3

Mesoporous silicon powder of 80 vol % porosity was investigated in connection with its ability to take up sebum. The mesoporous silicon was prepared by anodisation. The maximum oil uptake capacity was measured as the volume of oil needed to change the texture and consistency of the powder from dry clumps to a flowing, smooth paste. This point is known as the wet point, past which, oil which no longer fills the pores (because they are full), flows between the particles. This point is significant in cosmetic applications because more powder will need to be applied beyond this point to avoid the shiny appearance of facial sebum. If no additional powder is applied, the existing powder-oil layer on the face may start to lose adhesion and become uneven in texture. As a model for sebum, linseed oil uptake was tested in mesoporous silicon and compared with a commercially available powder containing silica and titanium dioxide. More specifically, the materials used were: anodised porous silicon (80 vol % porosity);

Sunjin SH219 porous silica and titanium dioxide powder; commercial raw linseed oil from Bartoline, Ltd; Ceraphyl 140A from International Specialty Products; Squalene from Sigma Aldrich; Corn Oil from Sigma Aldrich; Oleic Acid from Sigma Aldrich; Cholesterol from Sigma Aldrich; Cholesterol Palmitate from Sigma Aldrich; Dioleoylglycerol from Sigma Aldrich.

In order to determine linseed oil uptake, the wet point of each material was determined according to the procedure from Example 2, Section E of U.S. Pat. No. 6,730,309, the contents of which are hereby incorporated by reference in their entirety. Porous silicon was weighed into a glass jar. Linseed oil was added in increments of 0.2 g using a plastic pipette and mixed into the silicon between increments using a spatula. The wet point of the material was determined as the volume of oil added when the consistency of powder transitioned from dry clumps into a smooth paste. This procedure was repeated with the silica and titanium dioxide powder.

Artificial sebum was prepared in accordance with U.S. Pat. No. 4,515,784, the contents of which are hereby incorporated by reference in their entirety. The composition is as follows: squalene 18 wt %, corn oil 7 wt %, oleic acid 27 wt %, Ceraphyl 140A (decyl oleate) 43.5 wt %, cholesterol 2.5 wt %, cholesterol palmitate 1 wt %, glycerol dioleate: oleic acid (1:1) 1 wt %. The ingredients were combined in a glass jar and heated slightly above room temperature to facilitate mixing.

As for the linseed oil uptake, the wet point of the powders with artificial sebum was measured according to the procedure from Example 2, Section E of U.S. Pat. No. 6,730,309.

The wet point values for linseed oil in porous silicon and the porous silica/titanium powder are presented in Table 6.

TABLE 6
Linseed oil maximum loading capacities for various materials.

| | | Volume to Wet Point (ml/g) | |
|---|---|---|---|
| Adsorbent | Trial | Experimental Range | Average |
| Porous silicon powder | 1 | 1.90-2.11 | 2.01 |
| (80 μm diameter) | 2 | 1.88-2.09 | 1.99 |
| Porous silicon powder | 1 | 2.11-2.32 | 2.22 |
| (40 μm diameter) | | | |
| Sunjin SH219 silica | 1 | 0.61-0.82 | 0.72 |
| titanium powder | | | |
| (5 μm diameter) | | | |

Both sizes of porous silicon exhibited a significantly greater oil uptake capacity than the Sunjin silica titanium powder. Literature values for oil absorption of a similar powder produced by the same company (Sunsil 130) are 0.9-1.3 ml/g. No significant volume change was observed in the porous silicon after addition of the linseed oil. In contrast, the volume of the Sunjin powder visibly reduced after combination with the linseed oil.

Wet points for artificial sebum exhibit a similar trend as for the linseed oil wet points. Both porous silicon samples have greater than twice the oil capacity of the Sunjin silica titanium dioxide powder (Table 7).

TABLE 7
Artificial sebum maximum loading capacities for various materials.

| | | Volume to Wet Point (ml/g) | |
|---|---|---|---|
| Adsorbent | Trial | Experimental Range | Average |
| Porous silicon powder | 1 | 1.69-1.92 | 1.81 |
| (80 μm diameter) | 2 | 1.85-2.08 | 1.97 |
| Porous silicon powder | 1 | 1.76-2.14 | 1.95 |
| (40 μm diameter) | | | |
| Sunjin SH219 silica powder | 1 | 0.50-0.93 | 0.72 |
| (5 μm diameter) | 2 | 0.67-0.90 | 0.79 |

The results for artificial sebum uptake and linseed oil uptake are summarised in Table 8.

TABLE 8
Comparison of linseed oil and artificial sebum maximum loading
capacities for porous silicon and silica titanium dioxide.

| | | Volume to Wet Point (ml/g) | |
|---|---|---|---|
| | | Experimental | |
| Adsorbent | Solute | Range | Average |
| Porous | linseed oil | 1.88-2.11 | 2.00 |
| silicon powder | artificial sebum | 1.69-2.08 | 1.89 |
| (80 μm diameter) | | | |
| Porous silicon powder | linseed oil | 2.11-2.32 | 2.22 |
| (40 μm diameter) | artificial sebum | 1.76-2.14 | 1.95 |
| Sunjin SH219 | linseed oil | 0.61-0.82 | 0.72 |
| silica powder | artificial sebum | 0.67-0.90 | 0.79 |
| (5 μm diameter) | | | |

This example illustrates that both linseed oil and artificial sebum uptake capacities are significantly greater in porous silicon than the commercially available porous silica titanium dioxide powder.

1. A cosmetic composition for use on the human face comprising porous silicon.

2. A cosmetic composition according to claim 1, wherein the porous silicon may comprise at least one ingredient for delivery to the face.

3. A cosmetic composition according to claim 2, wherein the at least one ingredient is selected from one or more of: antioxidants, anti-ageing actives, skin lightening agents, nutrients, moisturisers, antimicrobials, fragrances, oils, vitamins, structural agents, natural actives.

4. A cosmetic composition according to claim 2, wherein the at least one ingredient is present in the range, in relation to the loaded porous silicon, of 0.01 to 60 wt %.

5. A cosmetic composition according to claim 1 wherein the porous silicon comprises, consists of, or consists essentially of mesoporous silicon.

6. A cosmetic composition according to claim 1, wherein the porous silicon comprises, consists of, or consists essentially of microporous silicon.

7. A cosmetic composition according to claim 1 wherein the porous silicon comprises modified surfaces.

8. A cosmetic composition according to claim 7 wherein the modified surfaces are selected from one or more of silicon hydride surfaces, silicon oxide surfaces, derivatised surfaces.

9. A cosmetic composition according to claim 1 wherein the porous silicon is capped with a capping layer.

10. A cosmetic composition according to claim 9 wherein the capping layer is selected from one or more of carbohydrates, gums, lipids, proteins, celluloses, synthetic polymers, synthetic elastomers, inorganic materials.

11. A cosmetic composition according to claim 9, wherein the capping layer is 300 nm to 500 nm thick.

12. A cosmetic composition according to claim 1 wherein the composition is selected from: foundation, mascara, lipstick, lip balm, lip gloss, colour cosmetics, face cream, eye cream, toner, shaving cream, after-shave, cleanser, aftersun, moisturiser, face masks, lip and eye liners, face powder, eye shadow, bronzer, blush, concealer, face scrub, make up remover.

13. A cosmetic composition according to claim 1 wherein the composition is in the form of one of the following: cream, paste, serum, gel, lotion, oil, milk, stick, ointment, powder, solution, suspension, dispersion, emulsion.

14. A cosmetic composition according to claim 1 wherein the particle size of the porous silicon is 5 μm to 250 μm.

15. A cosmetic composition according to claim 1, wherein the porous silicon is present in an amount of from 0.01 wt % to 40 wt % based on the total weight of the cosmetic composition.

16. A production process for forming the cosmetic composition according to claim 1, comprising blending said porous silicon and other components of the cosmetic composition.

17. A method of treating and/or cleaning the human face comprising applying a composition according to claim 1 to the human face.

18. A method according to claim 17 wherein the treatment is for the treatment or prevention of any one of acne, oily skin, wrinkles, psoriasis, birthmarks, scars, moles, blackheads, freckles, pimples, bags or dark circles under the eyes, rosacea, sebhorrhoeic dermatitis, enlarged pores, pitting, enlarged blood vessels, senile freckles.

19. A method according to claim 18 wherein the method is for treating the human face and the porous silicon adsorbs sebum.

20. A composition according to claim 1, wherein the composition is for use in the treatment or prevention of any one of acne, oily skin, wrinkles, psoriasis.

21. Use of porous silicon for delivering at least one active ingredient to the face.

22. Use of porous silicon according to claim 21 for delivering retinol to the face.

23. Use of porous silicon according to claim 22, wherein the porous silicon comprises an oxidised surface.

24. Use of porous silicon according to claim 21, wherein the porous silicon is mesoporous silicon.
